(19) United States
(12) Patent Application Publication
(10) Pub. No.: US 2019/0382331 A1
(43) Pub. Date: Dec. 19, 2019

(54) PROCESS FOR PREPARING BIS(2-HYDROXYETHYL) TEREPHTHALATE

(71) Applicant: Far Eastern New Century Corporation, Taipei City (TW)

(72) Inventors: Der-Ren Hwang, Taipei City (TW); Cheng-Ting Wang, Taipei City (TW); Hsiao-Chan Wang, Taipei City (TW)

(21) Appl. No.: 16/220,211

(22) Filed: Dec. 14, 2018

(30) Foreign Application Priority Data
Jun. 13, 2018 (TW) 107120286

(51) Int. Cl. C07C 67/26

(52) U.S. Cl. CPC C07C 67/26; C07C 69/82

(57) ABSTRACT

A process for preparing bis(2-hydroxyethyl) terephthalate, comprising a step of: subjecting ethylene oxide and terephthalic acid in a molar ratio of from 2.5:1 to 3.5:1 to a reaction at an elevated temperature of up to 150° C. in the presence of a solvent mixture containing water and a diol cosolvent in a weight ratio of from 0.2:1 to 5:1.

### CROSS-REFERENCE TO RELATED APPLICATION

This application claims priority of Taiwanese Application No. 107120286, filed on Jun. 13, 2018.

### FIELD

The disclosure relates to a process for preparing bis(2-hydroxyethyl) terephthalate, and more particularly to a process for preparing bis(2-hydroxyethyl) terephthalate at an elevated temperature in the presence of a solvent mixture containing water and a diol cosolvent.

### BACKGROUND

Bis(2-hydroxyethyl) terephthalate (referred to as BHET hereinafter) having Formula (I) as shown below is prepared industrially by subjecting ethylene oxide and terephthalic acid to a reaction.

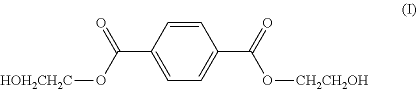

As shown by Formula (I), BHET has two hydroxyl end groups, and thus can be used as a diol component in the preparation of polyesters such as polyethylene terephthalate (PET).

U.S. Pat. No. 7,332,548 discloses a process to produce a partially esterified carboxylic acid product, which comprises contacting at least one dicarboxylic acid with at least one alkylene oxide in a reactor zone in the presence of at least one solvent and at least one basic catalyst. The solvent includes at least one selected from toluene and xylene. However, as shown in Table 5 of the published specification of U.S. Pat. No. 7,332,548, conversion rate of esterified terephthalic acid is only up to 54.4%, which indicates that the alkylene oxide such as ethylene oxide is not completely reacted. Therefore, a subsequent recycling treatment for residual alkylene oxide is required, resulting in increased production cost. Generally, the conversion rate of terephthalic acid should be higher than 90% for commercial mass production. In addition, reaction temperature for the process of producing the partially esterified carboxylic acid product is disadvantageously high, ranging from 180° C. to 280° C.

U.S. Pat. No. 6,310,233 discloses a process for reacting dicarboxylic acid with alkylene oxide to produce hydroxyalkyl ester monomers and perhaps minor amounts of oligomers using a mixture of water and dimethyl ether as a solvent. However, conversion rate of terephthalic acid in the process is generally less than 71% and a significant amount of mono (2-hydroxyethyl) terephthalate (referred to as MHET hereinafter) having Formula (II) as shown below is present in the obtained product.

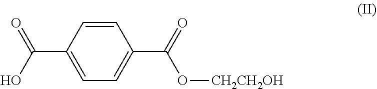

As shown by Formula (II), MHET has a hydroxyl end group and a carboxyl end group, and thus has an inferior reactivity in the preparation of polyesters, due to insufficient polymerization of the polyesters. It is desirable that in the process for preparing BHET, MHET is produced in an amount as low as possible so as to enhance subsequent process for preparation of the polyesters.

In addition, since organic solvent such as toluene or dimethyl ether is used in the aforesaid prior art, an extra time and costly treatment are required to remove the organic solvent as a waste.

### SUMMARY

An object of the disclosure is to provide a process for preparing bis(2-hydroxyethyl) terephthalate to overcome the aforesaid shortcomings.

According to an aspect of the disclosure, there is provided a process for preparing bis(2-hydroxyethyl) terephthalate, comprising a step of:
subjecting ethylene oxide and terephthalic acid in a molar ratio of from 2.5:1 to 3.5:1 to a reaction at an elevated temperature of up to 150° C. in the presence of a solvent mixture containing water and a diol cosolvent, in a weight ratio of from 0.2:1 to 5:1 to

### DETAILED DESCRIPTION

An embodiment of a process for preparing bis(2-hydroxyethyl) terephthalate according to the disclosure comprises a step of:
a) subjecting ethylene oxide and terephthalic acid in a molar ratio of from 2.5:1 to 3.5:1 to a reaction at an elevated temperature of up to 150° C. in the presence of a solvent mixture containing water and a diol cosolvent, in a weight ratio of from 0.2:1 to 5:1 to obtain a reaction mixture.

In certain embodiments, the molar ratio of the ethylene oxide to the terephthalic acid is in a range of from 2.5:1 to 3:1.

In certain embodiments, the elevated temperature is in a range of from 100° C. to 150° C. Specifically, the elevated temperature used in following examples is 120° C.

In certain embodiments, the diol cosolvent is selected from the group consisting of ethylene glycol, diethylene glycol, a diol compound of Formula (1), and combinations thereof,

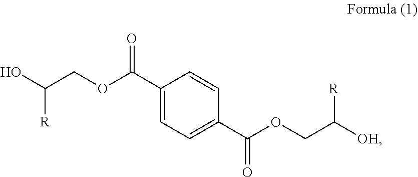

wherein R independently represents H or a C₁-C₆ linear or branched alkyl group.

In certain embodiments, R in Formula (1) independently represents H or a methyl group.

Specifically, the diol cosolvent used in some of the following examples is BHET (i.e., a diol compound of Formula (1) in which R is H).

In certain embodiments, the solvent mixture contains the water and the diol cosolvent in the weight ratio of from 0.2:1 to 1:1.

In certain embodiments, the solvent mixture contains the water and the diol cosolvent in the weight ratio of from 0.2:1 to 0.5:1.

In certain embodiments, after step a), the process for preparing bis(2-hydroxyethyl) terephthalate according to the disclosure further comprises a step of:
b) cooling the reaction mixture to a temperature of from 70° C. to 120° C.

In certain embodiments, after step a), the process for preparing bis(2-hydroxyethyl) terephthalate according to the disclosure further comprises steps of:
b) cooling the reaction mixture; and
c) removing the water from the reaction mixture at a temperature of from 75° C. to 120° C.

Examples of the disclosure will be described hereinafter. It is to be understood that these examples are exemplary and explanatory and should not be construed as a limitation to the disclosure.

### Example 1

Terephthalic acid (345 g, 2.077 mol), sodium carbonate (3.89 g), water (138 g), and ethylene glycol (69 g) were added into a 1 L stainless steel reactor (a total amount of water and ethylene glycol: 207 g), followed by heating under stirring to a temperature of 120° C. and then slowly adding ethylene oxide at a flow rate of 1 ml/min at a controlled temperature of 120° C. and under a controlled pressure of not above 7.0 kgf/cm² until 228.9 g (5.193 mol) of ethylene oxide was added. After continuous reaction for 15 minutes, the temperature was lowered to 80° C., the water was removed via distillation under a reduced pressure at the temperature of 80° C., and then cooling to room temperature was conducted to obtain a crude product containing BHET.

### Example 2

The procedures for obtaining a crude product of Example 2 were similar to those of Example 1 except that an added amount of ethylene oxide was 274.4 g (6.230 mol).

### Examples 3 to 5

The procedures for obtaining a crude product of each of Examples 3 to 5 were similar to those of Example 2 except that the amounts of water used in Examples 3 to 5 were 172.5 g, 69 g, and 34.5 g, respectively, and that the amounts of ethylene glycol used in Examples 3 to 5 were 34.5 g, 138 g, and 172.5 g, respectively. A total amount of water and ethylene glycol in each of Examples 3 to 5 was 207 g.

### Example 6

The procedures for obtaining a crude product of Example 6 were similar to those of Example 5 except that ethylene glycol used in Example 5 was replaced with diethylene glycol.

### Example 7

The procedures for obtaining a crude product of Example 7 were similar to those of Example 7 except that ethylene glycol used in Example 3 was replaced with BHET .

### Comparative Example 1

The procedures for obtaining a crude product of Comparative Example 1 were similar to those of Example 1 except that ethylene glycol was not added and that 207 g of water was used.

### Comparative Examples 2 and 3

The procedures for obtaining a crude product of each of Comparative Examples 2 and 3 were similar to those of Comparative Example 1 except that 182.9 g (4.153 mol) of ethylene oxide was added in Comparative Example 2 and that 274.4 g (6.230 mol) of ethylene oxide was added in Comparative Example 3.

### Comparative Example 4

The procedures for obtaining a crude product of Comparative Example 4 were similar to those of Example 1 except that 182.9 g (4.153 mol) of ethylene oxide was added.

The amounts of ethylene oxide, terephthalic acid, water, and a diol cosolvent (i.e., ethylene glycol, diethylene glycol, or BHET) used in each of Examples 1 to 7 and Comparative Examples 1 to 4 are summarized in Table 1 below.

TABLE 1

| | Reactants | | | Solvents | | |
|---|---|---|---|---|---|---|
| | Ethylene | Terephthalic | | Wa- | Diol | |
| | oxide | acid | Molar | ter | cosolvent | Weight |
| | (mol) | (mol) | ratio¹ | (g) | (g) | ratio² |
| Ex. 1 | 5.193 | 2.077 | 2.5:1 | 138 | Ethylene | 2:1 |
| | | | | | gylcol | |
| | | | | | 69 | |
| Ex. 2 | 6.230 | 2.077 | 3:1 | 138 | Ethylene | 2:1 |
| | | | | | gylcol | |
| | | | | | 69 | |
| Ex. 3 | 6.230 | 2.077 | 3:1 | 172.5 | Ethylene | 5:1 |
| | | | | | gylcol | |
| | | | | | 34.5 | |
| Ex. 4 | 6.230 | 2.077 | 3:1 | 69 | Ethylene | 0.5:1 |
| | | | | | gylcol | |
| | | | | | 138 | |
| Ex. 5 | 6.230 | 2.077 | 3:1 | 34.5 | Ethylene | 0.2:1 |
| | | | | | gylcol | |
| | | | | | 172.5 | |
| Ex. 6 | 6.230 | 2.077 | 3:1 | 34.5 | Diethylene | 0.2:1 |
| | | | | | gylcol | |
| | | | | | 172.5 | |
| Ex. 7 | 6.230 | 2.077 | 3:1 | 172.5 | BHET | 5:1 |
| | | | | | 34.5 | |
| Comp. | 5.193 | 2.077 | 2.5:1 | 207 | — | — |
| Ex. 1 | | | | | | |
| Comp. | 4.153 | 2.077 | 2:1 | 207 | — | — |
| Ex. 2 | | | | | | |
| Comp. | 6.230 | 2.077 | 3:1 | 207 | — | — |
| Ex. 3 | | | | | | |
| Comp. | 4.153 | 2.077 | 2:1 | 138 | Ethylene | 2:1 |
| Ex. 4 | | | | | glycol | |
| | | | | | 69 | |

Notes:
¹Molar ratio of ethylene oxide to terephthalic acid
²Weight ratio of water to diol cosolvent

### Analysis of Conversion Rate of Terephthalic Acid:

Conversion rate of terephthalic acid in the crude product of each of Examples 1 to 7 and Comparative Examples 1 to 4 was analyzed via ¹H NMR (300 MHz, solvent: dimethyl sulfoxide). The results are shown in Table 2 below.

### Analysis of MHET (Byproduct):

A molar ratio of MHET to BHET in the crude product of each of Examples 1 to 7 and Comparative Examples 1 to 4 was analyzed via HPLC (high performance liquid chromatography, solvent: a mixture of methanol with water in a weight ratio of 7:3; sample amount: 10 μl; wavelength: 254 nm; flow rate: 200 μl/min; three mobile phases in sequence: (1) a first mobile phase: a mixture of water with methanol in a volume ratio of 9:1 for 5 minutes, (2) a second mobile phase: a mixture of water with methanol in a linear gradient from a volume ratio of 9:1 to a volume ratio of 2:8 for 35 minutes, and (3) a third mobile phase: a mixture of water with methanol in a volume ratio of 2:8 for 5 minutes). The results are shown in Table 2.

TABLE 2

| | Conversion rate | |
|---|---|---|
| | of terephthalic | MHET/BHET |
| | acid | (molar ratio) |
| Ex. 1 | 90.06% | 6.6 × 10⁻² |
| Ex. 2 | 95.98% | 5.8 × 10⁻² |
| Ex. 3 | 94.42% | 6.8 × 10⁻² |
| Ex. 4 | 95.56% | 4.6 × 10⁻² |
| Ex. 5 | 91.56% | 2.3 × 10⁻² |
| Ex. 6 | 92.23% | 3.0 × 10⁻² |
| Ex. 7 | 95.97% | 5.8 × 10⁻² |
| Comp. Ex. 1 | 82.34% | 6.7 × 10⁻² |
| Comp. Ex. 2 | 83.65% | 8.9 × 10⁻² |
| Comp. Ex. 3 | 88.89% | 7.0 × 10⁻² |
| Comp. Ex. 4 | 83.61% | 7.4 × 10⁻² |

As shown in Table 2, in each of Examples 1 to 7, conversion rate of terephthalic acid is above 90% and a molar ratio of MHET/BHET is below 7.0×10⁻². Specifically, in each of Examples 4 to 6, a molar ratio of MHET/BHET is below 5.0×10⁻². However, in each of Comparative Examples 1 to 4, conversion rate of terephthalic acid is below 89% and a molar ratio of MHET/BHET is above 6.5×10⁻².

It should be noted that as shown in each of Examples 1 to 7, in the process for preparing bis (2-hydroxyethyl) terephthalate according to the disclosure, it is only necessary to remove water, which is used as a component of the solvent mixture , via distillation under a reduced pressure at a temperature of 80° C. Ethylene glycol, diethylene glycol, and/or BHET, which is used as another component of the solvent mixture, can remain in the reaction mixture and can be used as reactants in subsequent process for preparing polyesters such as polyethylene terephthalate (PET).

In view of the aforesaid, the process for preparing bis(2-hydroxyethyl) terephthalate according to the disclosure has advantages of a high conversion rate of terephthalic acid of at least 90% and a relatively low

MHET/BHET molar ratio of less than 7.0×10⁻². In addition, water, which is used as a component of the solvent mixture, can be removed easily via distillation under a reduced pressure at a temperature of 80° C. Ethylene glycol, diethylene glycol, and/or BHET, which is used as another component of the solvent mixture, can remain in the reaction mixture without incurring additional time and cost for removal and can be used as reactants for a subsequent process for preparing polyesters such as PET.

In the description above, for the purposes of explanation, numerous specific details have been set forth in order to provide a thorough understanding of the embodiment(s). It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A process for preparing bis(2-hydroxyethyl) terephthalate, comprising a step of:
a) subjecting ethylene oxide and terephthalic acid in a molar ratio of from 2.5:1 to 3.5:1 to a reaction at an elevated temperature of up to 150° C. in the presence of a solvent mixture containing water and a diol cosolvent, in a weight ratio of from 0.2:1 to 5:1 to obtain a reaction mixture.

2. The process according to claim 1, wherein the diol cosolvent is selected from the group consisting of ethylene glycol, diethylene glycol, a diol compound of Formula (1), and combinations thereof, 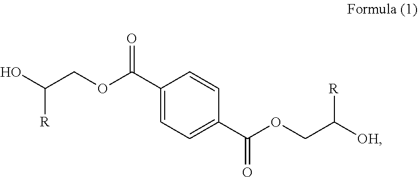
wherein R independently represents H or a C₁-C₆ linear or branched alkyl group.

3. The process according to claim 2, wherein R independently represents H or a methyl group.

4. The process according to claim 1, wherein the elevated temperature is in a range of from 100° C. to 150° C.

5. The process according to claim 1, wherein the solvent mixture contains the water and the diol cosolvent in the weight ratio of from 0.2:1 to 1:1.

6. The process according to claim 1, wherein the molar ratio of the ethylene oxide to the terephthalic acid is in a range of from 2.5:1 to 3:1.

7. The process according to claim 1, further comprising, after step a), a step of:
b) cooling the reaction mixture to a temperature of from 70° C. to 120° C.

8. The process according to claim 1, further comprising, after step a), steps of:
b) cooling the reaction mixture; and
c) removing water from the reaction mixture at a temperature of from 75° C. to 120° C.
